(19) United States
(12) Patent Application Publication
(10) Pub. No.: US 2024/0166849 A1
(43) Pub. Date: May 23, 2024

(54) ARYL ETHER DIAZIRINES FOR USE IN POLYMER CROSSLINKING AND ADHESION

(71) Applicant: XLYNX MATERIALS INC., Sidney (CA)

(72) Inventors: Jeremy WULFF, Victoria (CA); Peter BERRANG, Sidney (CA); Stefania MUSOLINO, Victoria (CA); Rashid NAZIR, Victoria (CA); Liting BI, Victoria (CA)

(73) Assignee: XLYNX MATERIALS INC., Sidney, BC (CA)

(21) Appl. No.: 18/279,167

(22) PCT Filed: Mar. 2, 2022
(86) PCT No.: PCT/CA2022/050293

§ 371 Date: Aug. 28, 2023

Related U.S. Application Data
(60) Provisional application No. 63/158,578, filed on Mar. 9, 2021.

(51) Int. Cl. C08K 5/3442; C07D 229/02; C07F 7/18; C08J 3/24; C08J 3/28; C08K 5/544

(52) U.S. Cl. CPC C08K 5/3442; C07D 229/02; C07F 7/1804; C08J 3/24; C08J 3/28; C08K 5/5477; C08J 2323/06

(57) ABSTRACT

A family of novel diazirine-based molecules is disclosed, as well as methods of manufacture and uses thereof. These compounds allow non-functionalized polymers, such as polyolefins, to crosslink via C—H insertion. Such a C—H insertion process is useful, for example, for the covalent adhesive bonding of low surface energy films or materials, or for creating rigid 3-dimenional polymeric structures by in-situ doping and activation of the crosslinker. The disclosed crosslinkers can be activated thermally, by UV radiation or by an electric potential.

### FIELD OF THE INVENTION

The invention relates to the field of crosslinking and adhesion of polymers, particularly of non-functionalized polymers.

### BACKGROUND OF THE INVENTION

Crosslinking of polymers increases mechanical strength and thermal stability, reduces material creep at high temperatures, provides resistance to electrical discharge, and offers increased stability to solvents and stress cracking. Moderate levels of crosslinking are known to be tolerated in many materials without the deleterious introduction of brittleness, and crosslinked polymers are already extensively used in everything from construction equipment to medical devices. However, the creation of interchain crosslinks generally requires that functional groups are already present within the polymer structure.

In cases where such functionality is absent, high energy processes (e.g. gamma-irradiation or introduction of free radicals) must be used to abstract hydrogen atoms. Such processes are expensive, non-tunable (tunability is the ability to rationally modify the properties of the final material by altering the properties and concentration of the crosslinker, or by altering the crosslinking conditions in a systematic way) and do not work for many industrially-important polymers (e.g. polypropylene) due to competing chain-fragmentation processes.

Addition of crosslinks to polymeric materials confers several important advantages to the final product. Impact resistance and tensile strength are increased, and material creep is vastly decreased. By fundamentally transforming a thermoplastic material into a thermoset, high temperature performance is greatly enhanced, and unwanted shrinkage at low temperature is reduced. Depending upon the nature and density of the chemical crosslinks, such materials often acquire shape memory, meaning that a deformed object will return to its original shape with the application of heat. These types of mechanical properties are required for many commercially important products.

Crosslinked materials also have increased resistance to solvents and electrical discharge, as well as to biological and chemical degradation. This is advantageous in applications where chemical-, biological- or electrically-promoted corrosion must be guarded against. Crosslinked polyethylene (“PEX”), for example, is used extensively for medical devices, as insulation for electrical wires, and for pipes used to transport corrosive liquids.

One potential disadvantage to crosslinking lies with an increase in brittleness. Because the polymer chains are no longer free to slip across each other, high-impact challenges can result in catastrophic material fracture. However, it is known in the art that, so long as the density of crosslinks is well controlled, brittleness can be avoided. Crosslinked polyethylene tubing, for example, typically has a crosslink density of 65-89%, while applications requiring greater flexibility have lower crosslink densities.

Crosslinks can be established in polymers in a number of ways. A common method for establishing a defined percentage of crosslinks involves first synthesizing a copolymer wherein one of the monomer constituents incorporates a linkable fragment. Such methods are not preferred industrially, as functionalized monomer components are often relatively expensive or difficult to synthesize. Further, the heavily functionalized copolymer often lacks the high strength or chemical resistance required for industrial applications.

Another strategy to achieve crosslinking involves the use of a monomer that has two functional groups—one to participate in the initial polymerization, and another to participate in subsequent crosslinking events. For example, in the industrially important thermoset material polydicyclopentadiene, one alkene in the monomer participates in the principal polymerization event, while a second alkene is largely responsible for crosslinking.

Unfortunately, neither of the above strategies is appropriate when one needs to crosslink an existing polymer material that has desirable properties (mechanical strength, ease of production, low cost, durability, etc.), but which lacks functionality within its chemical structure (“non-functionalized polymers”). This includes many extremely important industrial materials. For example, polyethylene (annual global production ca. 80 million tonnes), and polypropylene (ca. 55 million tonnes) are arguably the most important petrochemical-derived polymers on the planet, but do not easily lend themselves to chemical crosslinking. Similarly, biomass-derived polymers like polylactic acid and important biodegradable polymers like polycaprolactone often lack any crosslinkable functional groups, even though they contain some measure of functionality within their linear chains.

Existing methods for crosslinking non-functionalized polymers have a number of disadvantages. For example, crosslinked polyethylene can be produced by peroxide-initiated radical crosslinking. In this method, peroxide additives (e.g. dicumyl peroxide) are physically combined with polyethylene through an extrusion process. The resulting peroxide-impregnated polymer is then heated at high temperatures (typically 200-250° C.) to initiate the formation of radicals, which in turn results in abstraction of hydrogen atoms and eventual crosslinking. The key problem with prior art radical-based crosslinking methods is the need to break a very strong C—H bond, the strength for which is about 401 kJ/mol for the 2° C—H in polyethylene, and about 389 kJ/mol for the 3° C—H in polypropylene. Fundamentally, the need to generate such high-energy species as alkyl radicals means that little-to-no control is possible using crosslinking methods known in the prior art. Moreover, the carbon-centered radicals produced following cleavage of these strong C—H bonds are highly reactive and can undergo fragmentation (β-scission) reactions at rates that are competitive with crosslinking. This results in breakage of the polymer chains, and therefore reduces material strength.

Crosslinked polyethylene can also be produced by treatment with either gamma-rays or electron beams. As with peroxide crosslinking methods, these processes proceed via an initial cleavage of strong C—H bonds, and so suffer many of the disadvantages outlined above. The polymers produced using gamma-rays may, in some cases, have superior mechanical properties to those generated by peroxide-initiated methods, but the substantial costs associated with this process limits its use to the production of small-scale medical devices.

Both of the above methods (as well as related processes like silanization) generate intermediate radicals that can undergo β-scission and other undesirable side-reactions. β-Scission is reversible, and so tends not to be a limitation for crosslinked polyethylene (especially in high-density polyethylene); since the polymer chains are held close together, the products of radical fragmentation simply recombine to give the original secondary radical intermediate. For polypropylene, however, these types of processes are much more problematic.

A third problem with radical crosslinking is that the intermediates resulting from β-scission can recombine in a regiochemically different manner, ultimately leading to unexpected branching of the polymer structure. This can lead to a loss of crystallinity, and at the very least is hard to predict and control.

The crosslinking processes summarized above are not particularly tunable, beyond simple empirical controlling of total crosslink density. There is no provision for controlling the length or rigidity of the crosslink structure (which could be extremely useful in mitigating brittleness at high crosslink densities), nor is there any possibility to enhance the functionality of an existing polymer through these methods.

Indeed, given that isotactic polypropylene has higher mechanical strength than polyethylene (not to mention a higher melting point and better heat resistance), it is surprising that there is essentially no good crosslinking method available for polypropylene. This speaks not only to the significant limitations associated with radical-based crosslinking, but also suggests a significant untapped market for an eventual crosslinked polypropylene product.

Methods are known in the art for crosslinking polymers using diazirines. For example, Burgoon (US patent applications 20160083352 and 20180186747) discloses a family of diazirines useful as photocrosslinkers in the preparation of photoimageable compositions for film coating microelectronic or optoelectronic devices.

In general, the methods known in the art for application of diazirine-based crosslinkers all employ polymers that have existing functionality within their chemical structure beyond simple C—C and C—H bonds. The presence of such functionality either facilitates crosslinking or else lowers C—H bond strength (as is the case, for example, with polyether materials such as polyethylene oxide). Such functional polymers are generally intended for use in electronics applications (OLEDs, etc.).

WO/2020/215144 discloses a series of novel diazirines useful for crosslinking non-functionalized polymers, such as polyolefins. The compounds disclosed therein have advantages over methods of the art. They have a low barrier to C—H, O—H and N—H insertion, and they allow for the controllable crosslinking of essentially any polymer that contains C—H, O—H or N—H bonds. Moreover, such crosslinkers permit tunablity within the chemical structure of the crosslink itself.

As an added benefit, WO/2020/215144 teaches that diazirine-based crosslinkers can be used as adhesives. Painting (or otherwise applying) a quantity of crosslinker between two polymer objects and then activating the diazirine group can allow those skilled in the art to establish new bonds between the two polymer surfaces. The result of such new bonding is a strong adhesive force between the two objects.

It has now been discovered that the family of compounds disclosed herein have unexpected advantages over compounds of the art. Specifically, diazirines incorporating aryl ether linkages are found to be much more efficacious in reactions leading to insertions of unactivated C—H bonds, relative to previously studied molecular crosslinkers. In standard benchmarking experiments, the new aryl ether molecules displayed >10-fold improvement over a representative agent from WO/2020/215144. As an added benefit, the new aryl ether diazirines could be thermally activated at substantially lower temperatures than compounds known within the art, and have the potential to be activated at longer wavelengths of light.

### SUMMARY OF THE INVENTION

Compounds of Formula I, below, are useful as crosslinkers and adhesives, and are particularly useful for the crosslinking and adhesion of non-functionalized polymers.

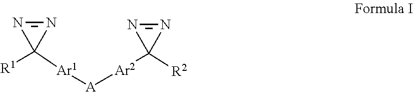

wherein:
A is selected from the group consisting of O, S and —X-L-Y—;
R¹ and R² are independently selected from the group consisting of alkyl and cycloalkyl;
Ar¹ and Ar² are independently selected from the group consisting of ortho-, meta- and para-phenylene;
X and Y are independently selected from the group consisting of O and S; and
L is a linear or branched divalent linker selected from the group consisting of saturated aliphatic chains and saturated ethers having from 2 to 20 carbon atoms.
L may include chemically or enzymatically cleavable motifs within the chain, such as esters, silyl ethers, peptides, and the like.

### DETAILED DESCRIPTION OF THE INVENTION

Disclosed are compounds of Formula I:

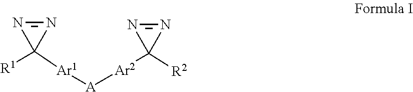

wherein:
A is selected from the group consisting of O, S and —X-L-Y—;
R¹ and R² are independently selected from the group consisting of alkyl and cycloalkyl;
Ar¹ and Ar² are independently selected from the group consisting of ortho-, meta- and para-phenylene;
X and Y are independently selected from the group consisting of O and S; and
L is a linear or branched divalent linker selected from the group consisting of saturated aliphatic chains and saturated ethers having from 2 to 20 carbon atoms.
L may include chemically or enzymatically cleavable motifs within the chain, such as esters, silyl ethers, peptides, and the like.

The term aryl ether, as used herein, includes both oxygen linkages and sulfur linkages (i.e. thioethers).

The term alkyl, as used herein, refers to alkyl groups having from 1 to 6 carbons, and includes both linear and branched groups. Non-limiting example of such groups includes methyl, ethyl, and isopropyl. Such alkyl groups may be halogenated. Non-limiting examples of halogenated alkyl groups includes fluoromethyl, difluoromethyl and trifluoromethyl. Preferably, R is a CF₃ group.

The term cycloalkyl, as used herein, refers to cycloalkyl groups having from 1 to 6 carbons, and includes cyclopropyl, cyclobutyl, cyclopentyl, cyclohexyl and the like. Such cycloalkyl groups may be halogenated. Non-limiting example of such groups include cyclopropyl and perfluorocyclopropyl.

The term saturated aliphatic chain, as used herein, includes ethylene, trimethylene, hexamethylene and the like.

The term saturated ether, as used herein, includes oligo(ethylene glycol) linkages (i.e., CH₂CH₂(OCH₂CH₂)ₙ), oligo(propylene glycol) linkages (i.e., CH₂CH₂CH₂(OCH₂CH₂CH₂)ₙ) and the like. It also includes S analogues such as CH₂CH₂(SCH₂CH₂)ₙ, CH₂CH₂CH₂SCH2CH2CH2)ₙ) and the like.

Compounds of Formula I wherein A is —X-L-Y— are preferred.

A preferred compound of Formula I is one in which R¹ and R² are CF₃, Arand Ar² are para-phenylene, X and Y are O, and is L is (CH₂)₈. A second preferred compound of Formula I is one in which R¹ and R² are CF₃, Ar¹ and Ar² are para-phenylene, X and Y are O, and is L is (CH₂)₁₄. A third preferred compound of Formula I is one in which R¹ and R² are CF₃, Ar¹ and Ar² are para-phenylene, X and Y are O, and L is C₂H₄OC₂H₄OC₂H₄OC₂H₄. A fourth preferred compound of Formula I is one in which R¹ and R² are CF₃, Ar¹ and Ar² are para-phenylene, X and Y are S, and is L is (CH₂)₈.

A fifth preferred compound of Formula I is one in which R¹ and R² are CF₃, Ar¹ and Ar² are para-phenylene, X and Y are O, and L is C₂H₄OSi(R³)₂OC₂H₄, where R³ is methyl, ethyl, isopropyl or tert-butyl. A sixth preferred compound of Formula I is one in which R¹ and R² are CF₃, Ar¹and Ar² are para-phenylene, X and Y are O, and L is C₂H₄OC(O)OC2H₄. A seventh preferred compound of Formula I is one in which R¹ and R² are CF₃, Ar¹ and Ar² are para-phenylene, X and Y are O, and L is C₂H₄O(CO)₂OC₂H₄. A further preferred compound of Formula I is one in which R¹ and R² are CF₃, Ar¹ and Ar² are para-phenylene, and A is O.

Certain compounds of Formula I have a relatively high N:C ratio. Although diazirines have been used for decades without incident, some simple diazirine compounds are known to present explosion hazards. Preferred compounds are those which have properties suitable for use in the processes described herein. Such compounds are not explosive, as determined by DSC data, impact tests, and Yoshida correlation analysis, as shown in Example 11.

Compounds of Formula I may be prepared using methods known in the art, as described herein. For example, they may be prepared by oxidation of a diaziridine precursor, which may in turn be obtained from the corresponding ketone or other suitable starting reagents. Examples 1-2 and 4-6 illustrate synthetic routes to prepare compounds of Formula I.

Compounds of Formula I are useful as crosslinkers, and have advantages over methods of the art. They have a low barrier to C—H, O—H and N—H insertion, and they allow for the controllable crosslinking of essentially any polymer that contains C—H, O—H or N—H bonds. Moreover, such crosslinkers permit tunablity within the chemical structure of the crosslink itself.

Without intending to limit the scope of the invention disclosed herein, it is thought that compounds of Formula I work by losing nitrogen to form reactive carbenes, which can then undergo C—H, O—H, or N—H insertion with polymers. This leads to chemical crosslinks. The crosslinking process can increase the material strength of the target polymer, increase the melting temperature, decrease solubility, etc. If two pieces of polymer have a layer of crosslinker applied between them, then the crosslinking process results in adhesion.

Without intending to limit the scope of the invention disclosed herein, it is thought that compounds of Formula I preferentially yield singlet carbenes upon loss of nitrogen, rather than triplet carbenes which may be preferentially obtained upon activation of other known diazirines.

Because these C—H insertion steps are nearly barrierless, they allow chemical crosslinking to proceed without p-scission or other fragmentation reactions taking place.

Moreover, the crosslinking process can take place with completely unfunctionalized polymers (e.g. polyethylene, polypropylene) as well as with other important polymers that contain functionality but are still not disposed toward crosslinking (e.g. polylactic acid, polycaprolactone). Compounds of Formula I also have advantages for polymers which can be crosslinked by more traditional methods (e.g. silicones), but for which there exist limitations with the current crosslinking technologies.

Compounds of Formula I can be activated thermally, photochemically, electrically, or through the use of transition metals. Surprisingly, thermal activation appears to be optimal for many applications; for example, in head-to-head crosslinking experiments of unfunctionalized materials (using cyclohexane as a model substrate) thermal activation is superior. By contrast, photochemical activation is the preferred method of activation for prior art methods using diazirines.

With respect to photochemical activation, diazirines can be activated at wavelengths beyond 254 nm, such as 360 or even 390 nm. This is well into the range at which most industrial polymers are optically clear, which means that loss of light to the bulk medium is minimal. Similarly, unwanted photo-degradation of the polymer substrate will be minimized by using these wavelengths, relative to radical-based processes which often require the use of high energy 254 nm light.

In principle, compounds of Formula I can be used to crosslink any organic polymer which has C—H or O—H or N—H bonds. Proof-of-concept experiments with polyethylene in Example 12 bear this out.

Thus, the chemical structure of various polymeric materials can be modified by the topical application or in-situ addition, and activation, of the crosslinkers described herein. Such chemical modification includes, for example, increasing the polymer's tensile strength, molecular weight, melting point, “stiffness”, and/or UV resistance and acting as an in-situ foaming agent.

Topical application of the crosslinkers described herein can also increase the polymer's surface energy, which is a key parameter that expands the commercial uses of such materials. Higher surface energy increases adhesion strength.

Selected polymer substrates include low surface energy materials having C—H bonds, such as, for example, polyethylene and polypropylene. Materials having O—H or N—H bonds are also applicable.

The format of such polymeric materials includes, for example, premade objects, films, powders, sheets, bare fibres, mesh and ribbons.

Such format materials can be further processed into shapes such as braided lines or ropes, woven and non-woven fabric, alternating orthogonal layers of unidirectional fibres, knitted fabric, laminated films and mesh or web constructs.

Powdered polymeric materials can also be sintered or pressure compacted into various shapes.

For materials comprised of woven or non-woven fibres, or braided lines or ropes, it may be advantageous to use a vacuum or high pressure to facilitate higher penetration of the crosslinker molecule and solvent carrier into such processed material.

The crosslinkers described herein can also be incorporated into the polymer material itself by, for example, by pressure or solvent infusion, where such infusion substantially disperses the crosslinker within the polymer.

Such infusion can be accomplished by dissolving the crosslinker in, for example, a volatile organic solvent such as pentane, (which can be removed prior to activation) at a temperature that does not melt the polymer or cause the crosslinker to activate. Optionally, a vacuum can be first applied to achieve higher crosslinker penetration in materials constructed of braided, woven and non-woven fibres, bare fibres or strands of fibres.

Alternatively, the crosslinker can be pressure infused with or without the use of a solvent carrier.

The addition of a crosslinker can also be accomplished by adding the crosslinker directly into the polymer melt or extrudant. Various applications of the crosslinkers disclosed herein are described below. These applications are not meant to be comprehensive but, rather, to illustrate some of the chemical modifications made possible by the addition of the disclosed crosslinkers.

### Application #1—Covalent Bonding of Polyolefin Films

The adhesive or thermal lamination of polyolefin and other polymer films is widely commercially available, especially in the food packaging industry. These packaging film laminates include a variety of high tensile strength biaxially oriented films.

However, bonding polyolefin films, especially PP (polypropylene) films, and BOPP (biaxially oriented polypropylene) is problematic, as peel strength (i.e. film bonding adhesion) requires prior surface treatment and is limited in peel strength between laminated films.

Also, polyethylene films, such as HDPE (high density polyethylene) and UHMWPE (ultra high molecular weight polyethylene) are difficult to strongly adhesively laminate.

The crosslinkers disclosed herein can be conveniently applied between selected films to be laminated using standard industry “glue” application processes, and then thermally activated to create a strong covalent bond.

Such covalently laminated films can be comprised of PE-to-PE (polyethylene-to-polyethylene), PP-to-PP (polypropylene-to-polypropylene) or PE-to-PP (polyethylene-to-polypropylene), where such covalent bonds are superior to prior art “glued” versions using specialized adhesives, as all glued together materials will ultimately fail, in time, if continually stress loaded. Also, moisture intrusion can act to de-laminate polymer film laminations, whereas covalently bonded interfaces are not subject to such delamination processes.

Application #2—Infusion of Crosslinkers into Polyolefin Films

Polyolefin films are used globally for packaging, including for food packaging.

The tensile strength, low surface energy, tear strength, gas diffusion and UV degradation are some of the key parameters which limit the use of these films.

Such parameters can be modified by the incorporating at least one of the crosslinkers disclosed herein into the material itself.

Application #3—Direct Addition of Crosslinkers into the Polymer Melt or Extrudant

Addition of one or more of the crosslinkers disclosed herein to a polymer melt or extrudant, followed by initiation of crosslinking by thermal, photochemical, or other means either during the extrusion process or following extrusion, can be used to control the material properties of the final polymer object.

Application #4—Pressure or Solvent Infusion of Crosslinkers into UHMWPE for Medical Implants

Shaped UHMWPE constructs are currently used as prostheses in medical implants. Such prior art implants have been modified using gamma-irradiation to increase material tensile strength. Such treatment using radiation is expensive, and thus limits widespread use.

Pressure or solvent infusion of at least one of the crosslinkers described herein, followed by low-temperature activation (<110° C.), provides a convenient, cost-effective method to modify such UHMWPE prostheses.

Application #5—Pressure or Solvent Infusion of Crosslinkers into UHMWPE Woven or Non-woven Fabrics and Related Materials

Such UHMWPE constructs, comprised of fibres braided into lines or ropes, and woven, non-woven or knitted articles, have a number of potential commercial applications.

For example, 100 gsm (gram per square metre) plain woven UHMWPE fabric, which has utility for ballistic protective garments, can be modified by the pressure or solvent infusion of at least one of the crosslinkers described herein which, when activated, can increase the effective tensile strength of the fabric. This significant increase in material tensile strength acts to increase the material ballistic resistance properties without significantly increasing material weight.

Besides ballistic applications, such crosslinker-modified woven fabric can also be used for articles such as high strength sails, tents, tarps, kites, carry bags, backpacks, etc.

### Application #6—3D-Printing

The 3D-printing of a wide array of articles using various thermoplastic polymers has expanded rapidly, with diverse applications of this elegant technology. However, the physical properties of such printed polymer articles are limited by the inherent properties of the polymers used during printing.

The opportunity to modify the physical properties of a printed polymer article by thermal activation, UV activation, or activation through the use of an applied electric field, using at least one of the crosslinkers disclosed herein, provides the user with heretofore new commercial possibilities.

### Application #7—Crosslinkers as a Natural Foaming Agent

The crosslinkers disclosed herein can act as natural foaming agents, due to the release of nitrogen gas during activation. When embedded into a polymer formulation of appropriate viscosity, activation of an appropriate amount of the crosslinker allows control of foaming parameters (expansion, density etc.).

Application #8—Polymers and Copolymers Derived from Crosslinkers

The crosslinkers disclosed herein can be activated in the absence of a polymer substrate to afford a polymeric material. Alternatively, the crosslinker can be combined with a suitable non-polymeric organic substrate (e.g. adamantane, mesitylene, tetramethylbiphenyl, tetrakis(p-tolyl)methane) or the like) prior to activation in order to form a network polymer.

### EXAMPLES

### Example 1: Synthesis of a Representative Aryl Ether Crosslinker with a Flexible Aliphatic Linker

### Step 1: Coupling of a Phenolic Precursor to a Suitable Linker

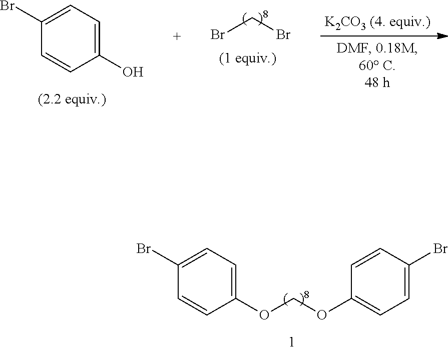

In a 1 L round bottom flask equipped with a magnetic stir bar and a condenser, to a stirring mixture of 4-bromophenol (14.8 g, 85.8 mmol, 2.2 equiv.) and potassium carbonate (21.5 g, 155.9 mmol, 4 equiv.) in DMF (200 mL), 1,8-dibromooctane (10.6 g, 38.9 mmol, 1 equiv.) was added. The mixture was heated to 60° C. for two days. The reaction mixture was cooled to room temperature, diluted with Et₂O followed by water, the aqueous layer was extracted with Et₂O (3 times) and EtOAc (1 time). The organic layers were combined, washed subsequently with brine, dried over Na₂SO₄, and concentrated in vacuo. The crude compound 1 was obtained as a white solid (17.6 g, 38.6 mmol, 99%). ¹H NMR (300 MHz, CDCl₃) δ 7.36 (d, J=9.0 Hz, 4H), 6.77 (d, J=8.9 Hz, 4H), 3.91 (t, J=6.5 Hz, 4H), 1.77 (dq, J=8.0, 6.4 Hz, 4H), 1.53-1.32 (m, 8H).

### Step 2: Installation of a Trifluoromethyl Ketone

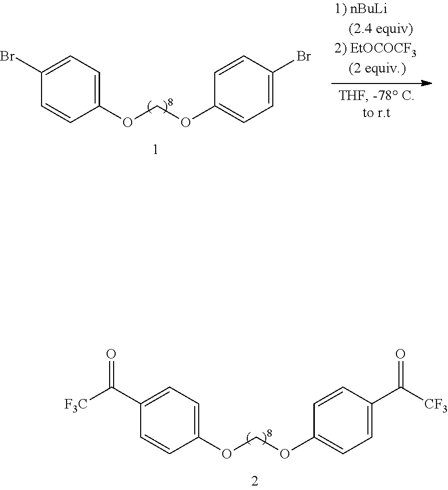

To a stirring solution of compound 1 (1.2 g, 2.6 mmol, 1 equiv.) in dry THF (15 mL) under argon atmosphere at −78° C., n-butyllithium (2.5 ml, 6.3 mmol, 2.4 equiv.) was slowly added and stirring was maintained at −78° C. for 1 h. Then ethyl trifluoroacetate (0.6 mL, 5.3 mmol, 2 equiv.) was added dropwise, and the mixture was stirred for a further 1 h at −78° C. and then allowed to warm to room temperature with continued stirring. After 6 h the reaction was quenched with sat. aq. NH₄Cl, and the aqueous layer was extracted with diethyl ether (3 times) and dried over MgSO₄. The dried organic layer was filtered and concentrated under reduced pressure. Flash-column chromatography over silica gel using petroleum ether:Et₂O (8:2) as eluent yielded 1.2 g (92%) of pure compound 2 as a white solid. ¹H NMR (500 MHz, CDCl₃) δ 8.04 (d, J=7.9 Hz, 4H), 6.98 (d, J=9.0 Hz, 4H), 4.07 (t, J=6.5 Hz, 4H), 1.88-1.79 (m, 4H), 1.56-1.46 (m, 4H), 1.41 (p, J=3.5 Hz, 4H). ¹³C NMR (126 MHz, CDCl₃) δ 165.15, 132.91, 122.77, 114.98, 68.67, 29.36, 29.11, 26.03. ¹⁹F NMR (283 MHz, CDCl₃) 67 -70.97.

### Step 3: Formation of a Bis-Oxime

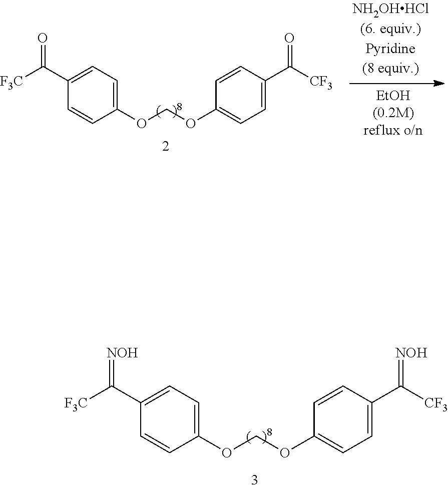

To a stirred solution of compound 2 (558 mg, 1.14 mmol, 1 equiv,) in ethanol (0.2 M), hydroxylamine hydrochloride (474 mg, 6.82 mmol, 6 equiv.) and pyridine (0.73 mL, 9.09 mmol, 8 equiv.) were added and the reaction mixture was heated to reflux for 16 h. The mixture was then cooled to room temperature and the mixture was treated with 2M HCl and extracted with Et₂O (3 times). The combined organic layers were washed with distilled water until the pH of the washing layer became neutral, and then dried with sodium sulfate, filtered and concentrated. The residue was dried under high vacuum for a prolonged time to afford the desired crude bis-oxime 3 (as a mixture of geometric isomers) as a white solid (505 mg). The compound was submitted to the next step without further purification. ¹H NMR (300 MHz, CDCl₃) δ 8.58 (s, 0.6H, minor isomer), 8.40 (d, J=16.7 Hz, 1H), 8.28 (d, J=10.1 Hz, 1H), 8.06 (s, 0.6H, minor isomer), 7.50 (d, J=8.5 Hz, 4H), 7.43 (d, J=8.4 Hz, 2.8H, minor isomer), 6.96 (d, J=8.9 Hz, 4H), 6.91 (d, J=8.6 Hz, 2.8H, minor isomer), 4.04-3.93 (m, 6.8H), 1.91-1.72 (m, 12H), 1.54-1.30 (m, 40H). ¹⁹F NMR (283 MHz, CDCl₃) δ −62.32, −66.26.

### Step 4: Activation of the Oxime by Formation of the Corresponding Nosylate

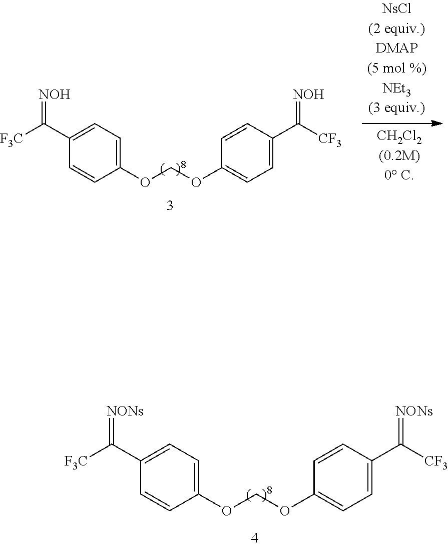

Compound 3 (505 mg, 0.97 mmol, 1 equiv.) was dissolved in CH₂Cl₂ (5 mL), and triethylamine (0.4 mL, 2.91 mmol, 3 equiv.), DMAP (6 mg, 0.048 mmol, 5 mol %) and nosyl chloride (430 mg, 1.94 mmol, 2 equiv.) were successively added at 0° C. The ice bath was removed after 5 min, and the reaction mixture was stirred at room temperature for 1 h. The mixture was then treated with sat. aq. NH₄Cl and extracted with CH₂Cl₂. The combined organic extracts were dried with magnesium sulfate, filtered, and concentrated to afford the desired crude bis-nosyloxime 4 (870 mg), which was submitted to the next step without further purification. ¹H NMR (300 MHz, CDCl₃) δ 8.28 (d, J=7.5 Hz, 2H), 7.95-7.77 (m, 4H), 7.61 (d, J=8.5 Hz, 4H), 7.00 (d, J=9.0 Hz, 4H), 4.02 (t, J=6.4 Hz, 4H), 1.92-1.70 (m, 14H), 1.53-1.29 (m, 35H). ¹⁹F NMR (283 MHz, CDCl₃) δ-65.60.

### Step 5: Formation of a Bis-Diaziridine

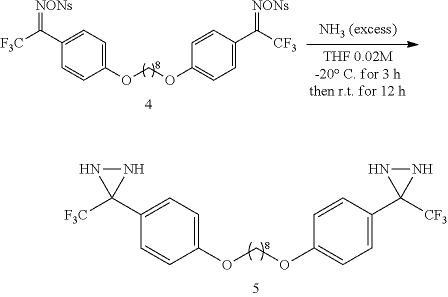

Bis-nosyloxime 4 (864 mg, 0.97 mmol, 1 equiv.) in anhydrous THF (20 mL) was transferred to a flame-dried 3-neck flask under argon and cooled to −20° C. Anhydrous gaseous ammonia was bubbled into the stirred solution for 3 h. Then, the reaction was left stirring for 12 h, during which time it was allowed to warm from −20° C. to room temperature. The mixture was quenched with sat. aq. NH₄Cl and extracted with Et₂O (3 times). The combined organic layers were washed with brine and then dried with magnesium sulfate, filtered and concentrated to afford the desired crude bis-diaziridine 5 (540 mg), which was submitted to the next step without further purification. ¹⁹F NMR (283 MHz, CDCl₃) δ −75.91.

### Step 6: Oxidation to the Desired Bis-Diazirine

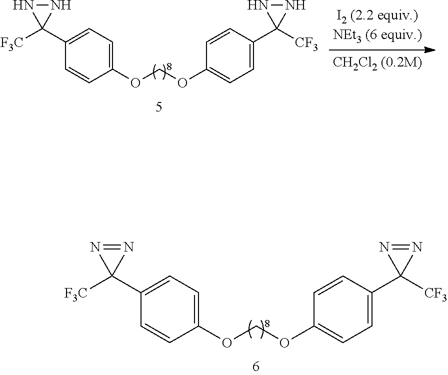

To a solution of the crude bis-diaziridine 5 (540 mg) in CH₂Cl₂ (5 mL) at 0° C. were added successively triethylamine (0.81 mL, 5.82 mmol, 6 equiv.) and iodine (542 mg, 2.13 mmol, 2.2 equiv.). The colored mixture was stirred at 0° C. for 1 h. The mixture was diluted with CH₂Cl₂ and washed with sat. aq. sodium thiosulfate. The aqueous layer was re-extracted with CH₂Cl₂ (3 times). Then the combined organic extracts were washed with brine and dried with magnesium sulfate, filtered, and concentrated. The residue was purified by silica gel column chromatography using pentane: Et₂O (8:2) as eluent to afford the desired bis-diazirine 6 (298 mg, 0.58 mmol, 60%) as a yellow solid. ¹H NMR (500 MHz, CDCl₃) δ 7.13 (d, J=8.4 Hz, 4H), 6.88 (d, J=8.9 Hz, 4H), 3.95 (t, J=6.5 Hz, 4H), 1.78 (p, J=6.6 Hz, 4H), 1.46 (dp, J=12.4, 6.5 Hz, 4H), 1.43-1.35 (m, 4H). ¹³C NMR (126 MHz, CDCl₃) δ 160.32, 129.55, 128.25, 123.50, 121.32, 120.85, 114.99, 68.22, 29.38, 29.23, 26.07. ¹⁹F NMR (283 MHz, CDCl₃) δ −65.63.

### Example 2: Synthesis of a Rigid Bis-Aryl Ether Crosslinker

### Step 1: Installation of a Trifluoromethyl Ketone

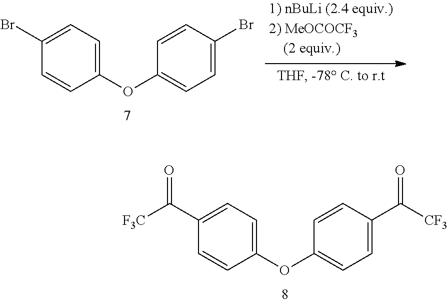 [SMILES: Brc1ccc(Oc2ccc(Br)cc2)cc1 ; O=C(c1ccc(Oc2ccc(C(=O)C(F)(F)F)cc2)cc1)C(F)(F)F]

To a stirring solution of 4,4′-oxybis(bromobenzene) 7 (636 mg, 1.94 mmol, 1 equiv.) in dry THF (50 ml) under argon atmosphere at −78° C., n-butyllithium (1.86 mL, 2.4 mmol, 2.5M) was slowly added and stirring was maintained at −78° C. for 1 h. Then methyl trifluoroacetate (0.40 mL, 3.9 mmol, 2 equiv.) was added dropwise, and the mixture was stirred for a further 1 h at −78° C. and then allowed to warm to room temperature with continued stirring. After 6 h the reaction was quenched with sat. aq. NH₄Cl, and the aqueous layer was extracted with Et₂O (3 times) and dried over MgSO₄. The dried organic layer was filtered and concentrated under reduced pressure. Flash-column chromatography over silica gel using petroleum ether: Et₂O (8:2) as eluent yielded 501 mg (62%) of pure compound 8 as a colorless oil. ¹H NMR (300 MHz, CDCl₃) δ 8.14 (d, J=8.0 Hz, 4H), 7.20 (d, J=8.9 Hz, 4H). ¹⁹F NMR (283 MHz, CDCl₃) δ −71.32.

### Step 2: Formation of a Bis-Oxime

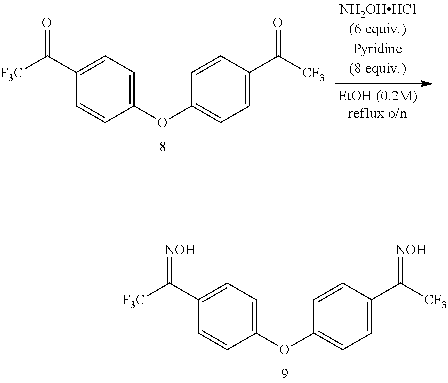

To a stirred solution of compound 8 (500 mg, 1.0 mmol, 1 equiv.) in ethanol (0.2 M), hydroxylamine hydrochloride (426 mg, 6.13 mmol, 6 equiv.) and pyridine (0.66 mL, 8.16 mmol, 8 equiv.) were added and the reaction mixture was heated to reflux for 16 h. The mixture was then cooled to room temperature and the mixture was treated with 2M HCl and extracted with Et₂O (3 times). The combined organic layers were washed with distilled water until the pH of the washing layer became neutral, and then dried with sodium sulfate, filtered and concentrated. The residue was dried under high vacuum for a prolonged time to afford the desired crude bis-oxime 9 (as a mixture of geometric isomers) (527 mg). The compound was submitted to the next step without further purification. ¹⁹F NMR (283 MHz, CDClI3) δ −62.26, −66.42.

### Step 3: Activation of the Oxime by Formation of the Corresponding Nosylate

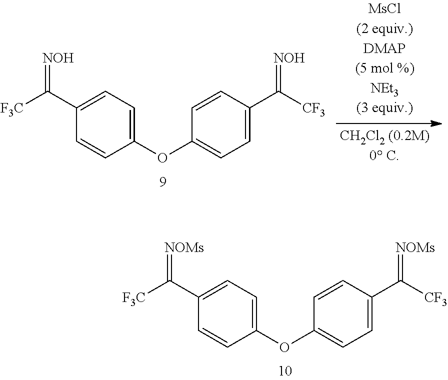

Compound 9 (527 mg, 1.1 mmol, 1 equiv.) was dissolved in CH₂Cl₂ (5 mL), and triethylamine (0.47 mL, 3.3 mmol, 3 equiv.), DMAP (6.8 mg, 0.056 mmol, 5 mol %) and mesyl chloride (0.17 mL, 2.24 mmol, 2 equiv.) were successively added at 0° C. The ice bath was removed after 5 min and the reaction mixture was stirred at room temperature for 2 h. The mixture was then treated with sat. aq. NH₄Cl and extracted with CH₂Cl₂. The combined organic extracts were dried with magnesium sulfate, filtered, and concentrated to afford the desired crude bis-mesyloxime 10 (596 mg) which was submitted to the next step without further purification. ¹H NMR (300 MHz, CDCl₃) δ 7.66 (d, J=8.9 Hz, 4H), 7.58 (dd, J=9.0, 2.9 Hz, 4H), 7.23-7.10 (m, 8H), 3.29 (s, 6H), 3.27 (s, 6H). ¹⁹F NMR (283 MHz, CDCl₃) δ −61.39, −66.22.

### Step 4: Formation of a Bis-Diaziridine

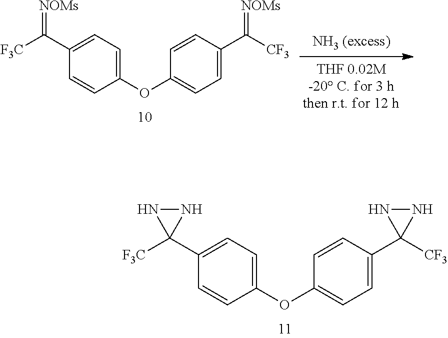

Bis-mesyloxime 10 (596 mg, 1.0 mmol, 1 equiv.) in anhydrous THF (20 mL) was transferred to a flame-dried 3-neck flask under argon and cooled to −20° C. Anhydrous gaseous ammonia was bubbled into the stirred solution for 3 h. Then, the reaction was left stirring for 12 h, during which time it was allowed to warm from −20° C. to room temperature. The mixture was quenched with sat. aq. NH₄Cl and extracted with Et₂O (3 times). The combined organic layers were washed with brine and then dried with magnesium sulfate, filtered and concentrated to afford the desired crude bis-diaziridine 11 (290 mg), which was submitted to the next step without further purification. ¹⁹F NMR (283 MHz, CDCl₃) δ −75.81.

### Step 5: Oxidation to the Desired Bis-Diazirine

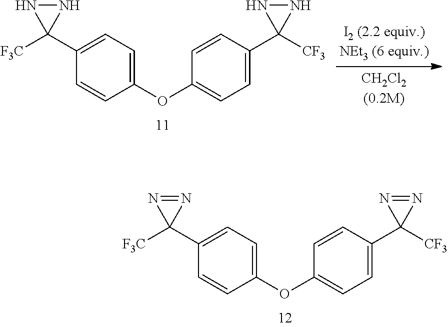 [SMILES: FC(F)(F)C1(c2ccc(Oc3ccc(C4(C(F)(F)F)NN4)cc3)cc2)NN1 ; FC(F)(F)C1(c2ccc(Oc3ccc(C4(C(F)(F)F)N=N4)cc3)cc2)N=N1]

To a solution of the crude bis-diaziridine 11 (276 mg) in CH₂Cl₂ (5 mL) at 0° C. were added successively triethylamine (0.6 mL, 4.24 mmol, 6 equiv.) and iodine (394 mg, 1.5 mmol, 2.2 equiv.). The colored mixture was stirred at 0° C. for 1 h. The mixture was diluted with CH₂Cl₂ and washed with sat. aq. sodium thiosulfate. The aqueous layer was re-extracted with CH₂Cl₂ (3 times). Then the combined organic extracts were washed with brine and dried with magnesium sulfate, filtered and concentrated. The residue was purified by silica gel column chromatography using pentane as eluent to afford the desired bis-diazirine 12 (244 mg, 0.63 mmol, 63%) as a colourless liquid. ¹H NMR (300 MHz, CDCl₃) δ 7.20 (d, J=8.8 Hz, 4H), 7.01 (d, J=8.9 Hz, 4H). ¹³C NMR (126 MHz, CDCl₃) δ 157.79, 128.67, 124.52, 119.45, 29.86. ¹⁹F NMR (283 MHz, CDCl₃) δ −65.45.

### Example 3: Synthesis of a Useful Building Block for Preparing Cleavable Crosslinkers and Related Diazirine-Containing Reagents

### Step 1: Iodination of Trifluoromethyl Phenyldiazirine

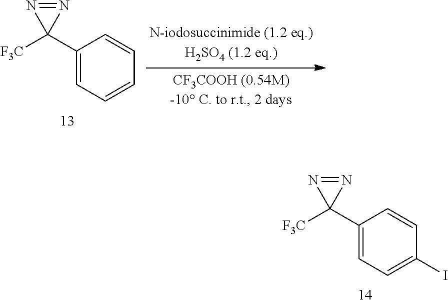 [SMILES: FC(F)(F)C1(c2ccccc2)N=N1 ; FC(F)(F)C1(c2ccc(I)cc2)N=N1]

To a solution of diazirine 13 (5.0 g, 27 mmol, 1.0 equiv.) in trifluoroacetic acid (TFA, 30 mL) at −10° C., N-iodosuccinimide (7.3 g, 32 mmol, 1.2 equiv.) was added portion-wise at −10° C. under ambient atmosphere. After the added N-iodosuccinimide had fully dissolved, a solution of H₂SO₄ (1.7 mL, 32 mmol, 1.2 equiv.) in trifluoroacetic acid (20 mL) was added dropwise into the reaction mixture. An empty balloon was added to facilitate the escape of any gas that was generated. The reaction was slowly warmed to room temperature and stirred for two days. The mixture was then poured into saturated sodium bicarbonate solution at 0° C. and extracted with diethyl ether (×3). The combined organic layers were washed with sodium thiosulfate and dried over sodium sulfate. After evaporation of the solvent, the residue was purified by silica gel column chromatography (using 100% pentane as an eluent) to afford the desired pure product as a yellow oil (6.1 g, 20 mmol, 74%, yellow solid at −20° C.). ¹H NMR (500 MHz, CDCl₃) δ 7.74 (d, J=8.6 Hz, 2H), 6.93 (d, J=8.2 Hz, 2H). ¹C NMR (126 MHz, CDCl₃) δ 138.18, 128.91, 128.25, 122.05 (q, J=274.7 Hz), 96.12, 77.16, 28.28. ¹⁹F NMR (471 MHz, CDCl₃) δ −65.26.

### Step 2: Addition of an Ethylene Glycol Handle Through Copper Coupling

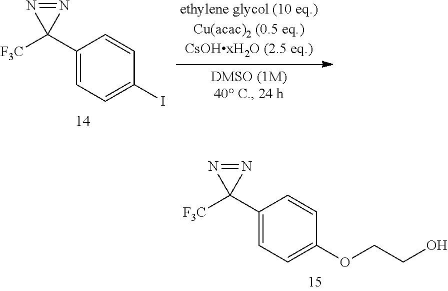 [SMILES: FC(F)(F)C1(c2ccc(I)cc2)N=N1 ; OCCOc1ccc(C2(C(F)(F)F)N=N2)cc1]

To a solution of diazirine 14 (1.5 g, 4.8 mmol, 1.0 equiv.) in DMSO (4.5 mL, 1.0 M) at room temperature, ethylene glycol (2.7 mL, 48 mmol, 10 equiv.) was added under ambient atmosphere. Then, Cu(acac)₂ (0.63 g, 2.4 mmol, 0.5 equiv.) was added, followed by CsOH⋅xH₂O (2.0 g, 12 mmol, 2.5 equiv.). The reaction mixture was warmed to 40° C. and stirred at that temperature for 24 hours. The mixture was then poured into water and extracted with dichloromethane (×3). The combined organic layers were dried over sodium sulfate. After evaporation of the solvent, the residue was purified by silica gel column chromatography (eluting with 0-100% ethyl acetate in pentane) to afford the desired pure product as a yellow oil (0.45 g, 1.8 mmol, 38%, yellow solid at −20° C.). ¹H NMR (500 MHz, CDCl₃) δ 7.15 (d, J=8.6 Hz, 2H), 6.92 (d, J=8.9 Hz, 2H), 4.12-4.04 (m, 2H), 4.02 - 3.93 (m, 2H), 2.04 (m, 1H). ¹³C NMR (126 MHz, CDCl₃) δ 159.81, 128.36, 122.36 (q, J=274.4 Hz), 121.58, 115.07, 69.47, 61.44, 28.35 (q, J=38.8 Hz). ¹⁹F NMR (471 MHz, CDCl₃) δ −65.63.

### Example 4: Synthesis of a Silyl Ether-Containing Crosslinker

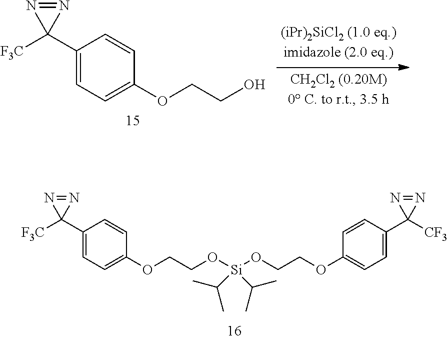 [SMILES: OCCOc1ccc(C2(C(F)(F)F)N=N2)cc1 ; CC(C)[Si](OCCOc1ccc(C2(C(F)(F)F)N=N2)cc1)(OCCOc1ccc(C2(C(F)(F)F)N=N2)cc1)C(C)C]

In a flame-dried round bottom flask under an atmosphere of argon, imidazole (0.028 g, 0.41 mmol, 2.0 equiv.) was added followed by dropwise addition of dichlorodiisopropylsilane (0.024 mL, 0.20 mmol, 1.0 equiv.) into a solution of diazirine 15 (0.10 g, 0.41 mmol, 2.0 equiv.) in anhydrous dichloromethane (2.0 mL, 0.20 M) at 0° C. After stirring for 3.5 hours at room temperature while shielding from light, the reaction mixture was filtered and washed with dichloromethane. After evaporation of the solvent, the residue was purified by eluting through a plug of silica gel (with 100% pentane) to afford the desired pure product as a yellow oil (0.062 g, 0.10 mmol, 50%, pale yellow solid at −20° C.). ¹H NMR (500 MHz, CDCl₃) δ 7.12 (d, J=8.6 Hz, 4H), 6.89 (d, J=8.8 Hz, 4H), 4.16-4.00 (m, 8H), 1.05 (s, 14H). ¹³C NMR (126 MHz, CDCl₃) δ 160.11, 128.26, 122.40 (d, J=274.8 Hz), 121.20, 115.07, 77.16, 69.47, 61.68, 28.36 (q, J=39.6 Hz), 17.31, 12.19. ¹⁹F NMR (471 MHz, CDCl₃) δ −65.65.

### Example 5: Synthesis of a Carbonate-Containing Crosslinker

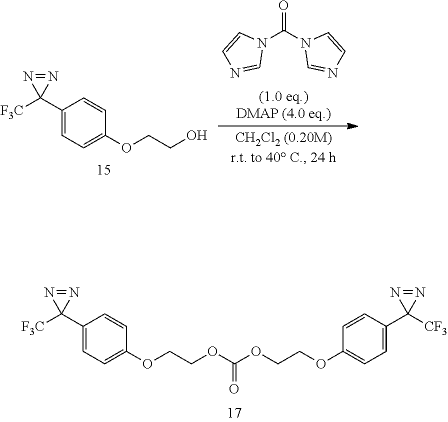 [SMILES: O=C(n1ccnc1)n1ccnc1 ; OCCOc1ccc(C2(C(F)(F)F)N=N2)cc1 ; O=C(OCCOc1ccc(C2(C(F)(F)F)N=N2)cc1)OCCOc1ccc(C2(C(F)(F)F)N=N2)cc1]

To a solution of diazirine 15 (0.050 g, 0.20 mmol, 1.0 equiv.) in dichloromethane (1.0 mL, 0.20 M) at room temperature, DMAP (0.099 g, 0.81 mmol, 4.0 equiv.) was added followed by 1,1′-carbonyldiimidazole (0.33 g, 0.20 mmol, 1.0 equiv.) under ambient atmosphere.

After three hours stirring at room temperature, the second equivalent of diazirine 15 (0.050 g, 0.20 mmol, 1.0 equiv.) was added directly into the reaction mixture. The mixture was warmed to 40° C. and stirred overnight. The reaction was then quenched by addition of a 0.25 M HCl aqueous solution and extracted with dichloromethane (×3). The combined organic layers were dried over sodium sulfate. After evaporation of the solvent, the residue was purified by silica gel column chromatography (eluting with 0-100% ethyl acetate in pentane) to afford the desired pure product as a yellow oil (0.080 g, 0.15 mmol, 75%, yellow oil at −20° C.). ¹H NMR (500 MHz, CDCl₃) δ 7.14 (d, J=8.7 Hz, 4H), 6.90 (d, J=8.9 Hz, 4H), 4.59-4.45 (m, 4H), 4.28-4.16 (m, 4H). ¹³C NMR (126 MHz, CDCl₃) δ 159.47, 155.03, 128.33, 122.35 (q, J=274.5 Hz), 121.76, 115.09, 66.23, 65.83, 28.32 (q, J=40.4 Hz). ¹⁹F NMR (471 MHz, Chloroform-d) δ −65.63.

### Example 6: Synthesis of an Oxalate-Containing Crosslinker

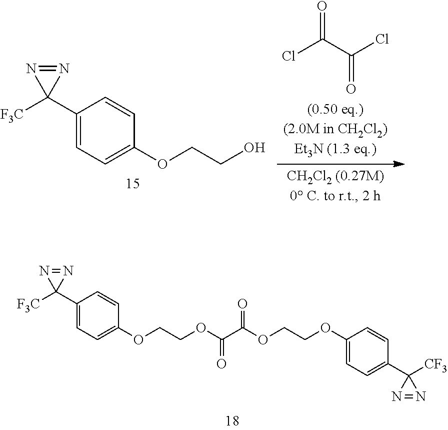 [SMILES: OCCOc1ccc(C2(C(F)(F)F)N=N2)cc1 ; O=C(Cl)C(=O)Cl ; O=C(OCCOc1ccc(C2(C(F)(F)F)N=N2)cc1)C(=O)OCCOc1ccc(C2(C(F)(F)F)N=N2)cc1]

In a flame-dried round bottom flask under an atmosphere of argon, triethylamine (0.071 mL, 0.51 mmol, 1.3 equiv.) was added followed by dropwise addition of oxalyl chloride (0.10 mL, 0.20 mmol, 0.50 equiv., 2.0 M in dichloromethane) into a solution of diazirine 15 (0.10 g, 0.41 mmol, 1.0 equiv.) in anhydrous dichloromethane (1.5 mL, 0.27 M) at 0° C. After stirring for 2 hours at room temperature, the reaction mixture was directly connected to high vacuum for a prolonged time to afford the crude mixture. Then the solid crude product was filtered and washed with cold diethyl ether to afford the desired pure product as a white solid (0.092 g, 0.17 mmol, 85%). ¹H NMR (500 MHz, CDCl₃) δ 7.15 (d, J=8.7 Hz, 4H), 6.90 (d, J=8.9 Hz, 4H), 4.70 -4.60 (m, 4H), 4.32-4.21 (m, 4H). ¹³C NMR (126 MHz, CDCl₃) δ 159.34, 157.30, 128.39, 122.33 (q, J=274.5 Hz), 121.95, 115.15, 65.40, 65.06, 28.30 (q, J=40.7 Hz). ¹⁹F NMR (471 MHz, CDCl₃) δ −65.63.

### Example 7: Evaluation of Crosslinker Efficacy by Crosslinking of Cyclohexane

It is known in the art that cyclohexane can be useful as a molecular model of polyethylene, in that both molecules can be said to comprise an infinite chain of CH₂ groups. By thermally activating various bis-diazirines in cyclohexane and then isolating the products of C—H insertion, one can gain insight into which species are most effective for the crosslinking of low-functionality polymers such as polyethylene or polypropylene.

Surprisingly, this exercise revealed that bis-diazirine 6, in which the aryl diazirine groups are separated by an aliphatic tether, was >10-fold more effective at crosslinking cyclohexane than were previously studied molecular crosslinkers. Rigid diaryl ether 12, in which the two aryl diazirine groups are bridged by a single oxygen atom, was also more effective than previously described molecular crosslinkers, but was less effective than bis-ether 6.

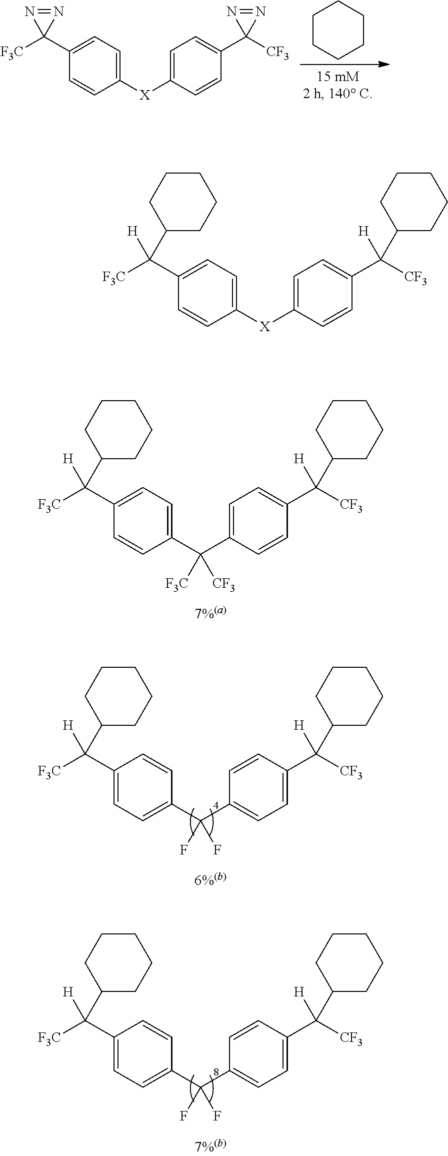

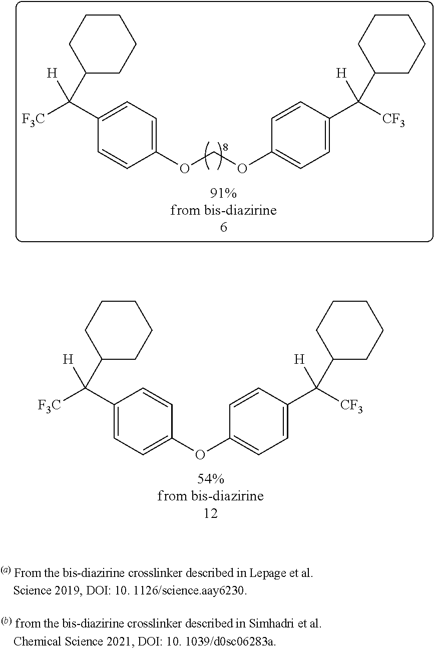

### Example 8: Cyclohexane Crosslinking with Bis-Diazirine 6

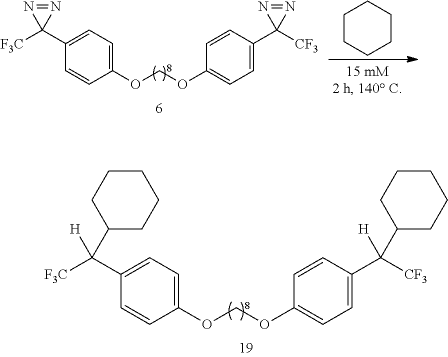

In a flame-dried sealed tube, bis-diazirine 6 (11.3 mg, 0.022 mmol, 1 equiv.) in cyclohexane (15 mM), flushed gently with argon and capped, was heated at 140° C. for 2 h. After cooling the mixture to room temperature, the reaction mixture was transferred into a round bottom flask and concentrated in vacuo to provide crude product (14 mg). Flash-column chromatography over silica gel using 100% petroleum ether afforded compound 19 (12 mg, 0.02 mmol) in 91% yield. ¹H NMR (300 MHz, CDCl₃) δ 7.05 (d, J=8.6 Hz, 4H), 6.78 (d, J=8.7 Hz, 4H), 3.87 (t, J=6.5 Hz, 4H), 2.90 (qd, J=10.3, 7.9 Hz, 2H), 1.95-1.79 (m, 4H), 1.77-1.62 (m, 8H), 1.61-1.50 (m, 4H), 1.45-1.27 (m, 12H), 1.11-0.96 (m, 4H), 0.74 (dd, J=12.1, 3.0 Hz, 2H). ¹³C NMR (126 MHz, CDCl₃) δ 158.64, 132.21, 130.20, 126.98, 116.30, 114.31, 67.87, 55.41, 55.21, 38.54, 31.53, 30.68, 29.72, 29.32, 29.29, 26.20, 26.12, 26.04. ¹⁹F NMR (283 MHz, CDCl₃) δ −63.73.

### Example 9: Cyclohexane Crosslinking with Bis-Diazirine 12

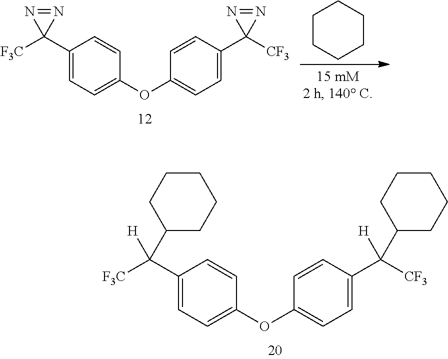

In a flame-dried sealed tube, bis-diazirine 12 (24.9 mg, 0.064 mmol, 1 equiv.) in cyclohexane (15 mM), flushed gently with argon and capped, was heated at 140° C. for 2 h. After cooling the mixture to room temperature, the reaction mixture was transferred into a round bottom flask and concentrated in vacuo to provide crude product (34.6 mg). Flash-column chromatography overs silica gel 100% petroleum ether, afforded compound 20 (17.6 mg, 0.034 mmol) in 54% yield. ¹H NMR (300 MHz, CDCl₃) δ 7.20 (d, J=8.7 Hz, 4H), 6.98 (d, J=8.7 Hz, 3H), 3.03 (p, J=9.8 Hz, 2H), 2.03-1.84 (m, 5H), 1.84-1.71 (m, 2H), 1.68-1.59 (m, 5H), 1.22-1.01 (m, 8H), 0.85-0.72 (m, 2H). ¹³ C NMR (76 MHz, CDCl₃) δ 130.69, 118.91, 38.68, 30.84, 26.23, 26.14. ¹⁹F NMR (283 MHz, CDCl₃) δ 63.60.

### Example 10: Assessment of Thermal Parameters for Representative Aryl Ether Crosslinkers

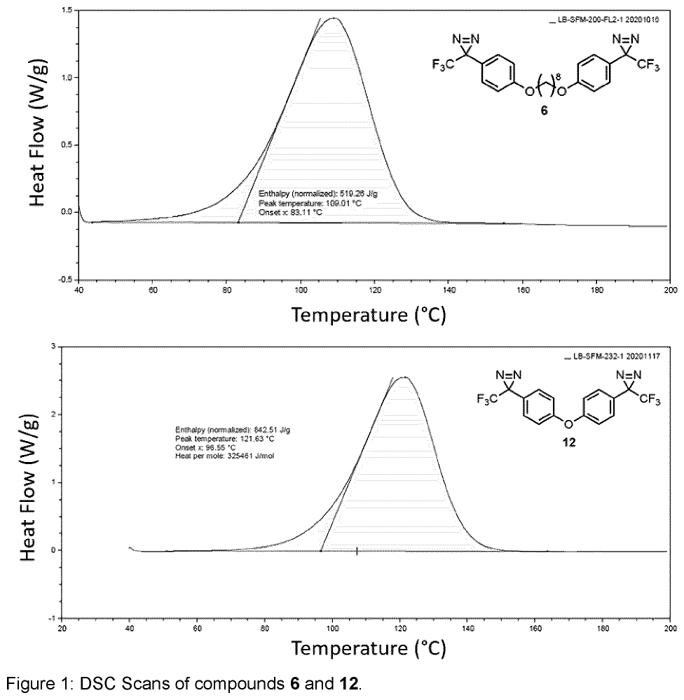

It is known in that art that differential scanning calorimetry (DSC) can be used to assess the thermal activation temperatures for bis-diazirine crosslinkers. DSC scans for compounds 6 and 12 are shown in FIG. 1. Surprisingly, bis-diazirine 6, in which the aryl diazirine groups are separated by an aliphatic tether, could be activated at approximately 30° C. lower than previously studied molecular crosslinkers. Rigid diaryl ether 12, in which the two aryl diazirine groups are bridged by a single oxygen atom, could also be activated at a lower temperature than previously described molecular crosslinkers, but required a higher temperature than compound 6.

Crosslinkers that activate at lower temperatures may be advantageous for crosslinking commodity polymers, or for adhesion applications using those same polymers, when one needs to avoid surpassing the melting temperature of the polymer substrate.

| | | Tₘₐₓ |
|---|---|---|
| Crosslinker | Tₒₙₛₑₜ | (° C.) |
| 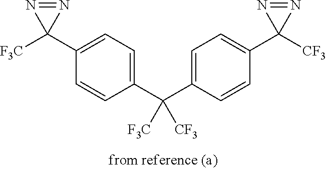 [SMILES: FC(F)(F)C1(c2ccc(C(c3ccc(C4(C(F)(F)F)N=N4)cc3)(C(F)(F)F)C(F)(F)F)cc2)N=N1] | 113° C. | 137° C. |
| 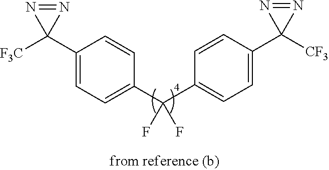 | 117° C. | 142° C. |
| 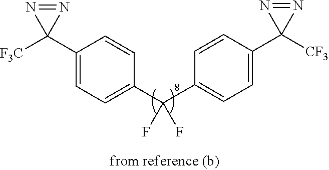 | 117° C. | 143° C. |
| 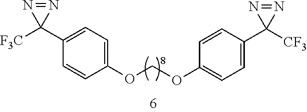 | 83° C. | 108° C. |
| 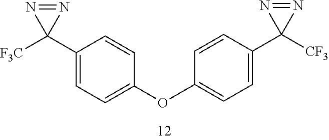 [SMILES: FC(F)(F)C1(c2ccc(Oc3ccc(C4(C(F)(F)F)N=N4)cc3)cc2)N=N1] | 96° C. | 122° C. |

(a) Lepage et al. Science 2019, DOI: 10.1126/science.aay6230.
(b) Simhadri et al. Chemical Science 2021, DOI:10.1039/d0sc06283a.

### Example 11: Assessment of Explosivity

It is known in the art that certain formulae developed by Yoshida can be used to predict the likely propensity for explosive propagation or shock sensitivity for a compound that contains multiple nitrogen atoms:

Shock sensitivity=log(Q_{DSC})−0.72×log(Tₒₙₛₑₜ−25)−0.98

Explosive propagation=log(Q_{DSC})−0.38×log(Tₒₙₛₑₜ−25)−1.67

where Q_{DSC} is the enthalpy of nitrogen release (in cal/g) and Tₒₙₛₑₜ is the onset temperature for diazirine activation (measured by extrapolation of the tangent of the DSC curve, and reported in ° C.). A material is considered likely to be explosive if shock sensitivity and/or explosive propagation values are positive.

| | Q_{DSC} | Q_{DSC} | | |
|---|---|---|---|---|
| Crosslinker | (J/g) | (kJ/mol) | SS | EP |
| 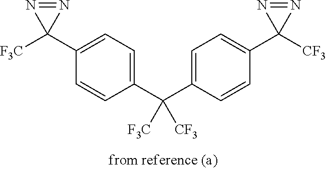 [SMILES: FC(F)(F)C1(c2ccc(C(c3ccc(C4(C(F)(F)F)N=N4)cc3)(C(F)(F)F)C(F)(F)F)cc2)N=N1] | 646 | 335 | −0.19 | −0.22 |
| 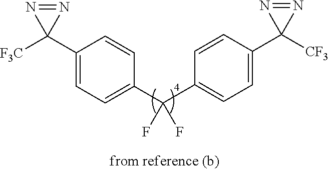 | 587 | 335 | −0.24 | −0.27 |
| 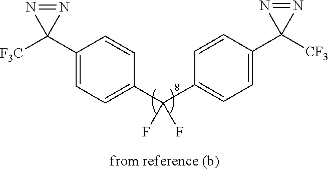 | 430 | 331 | −0.38 | −0.40 |
| 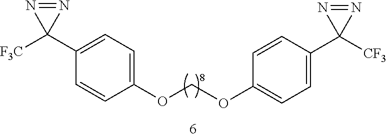 | 628 | 323 | −0.07 | −0.16 |
| 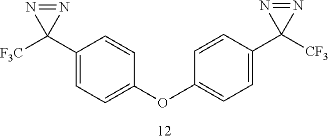 [SMILES: FC(F)(F)C1(c2ccc(Oc3ccc(C4(C(F)(F)F)N=N4)cc3)cc2)N=N1] | 837 | 324 | −0.01 | −0.07 |

(a) Lepage et al. Science 2019, DOI: 10.1126/science.aay6230.
(b) Simhadri et al. Chemical Science 2021, DOI:10.1039/d0sc06283a.

As a result of the increased mass present in the aliphatic tether group, bis-diazirine 6 released significantly less energy per unit weight than did diaryl ether 12, in which the two aryl diazirine groups are bridged by a single oxygen atom.

Surprisingly, the result of this reduction in energy output upon thermal activation is that bis-ether 6—despite its reduced activation temperature relative to previously studied molecular crosslinkers—was not found through the application of Yoshida's correlations to be a likely shock-sensitive reagent, or to be likely to sustain an explosion. By contrast, diaryl ether 12 is a potentially shock-sensitive reagent, since calculation of its shock sensitivity score results in a value that is within statistical range of zero.

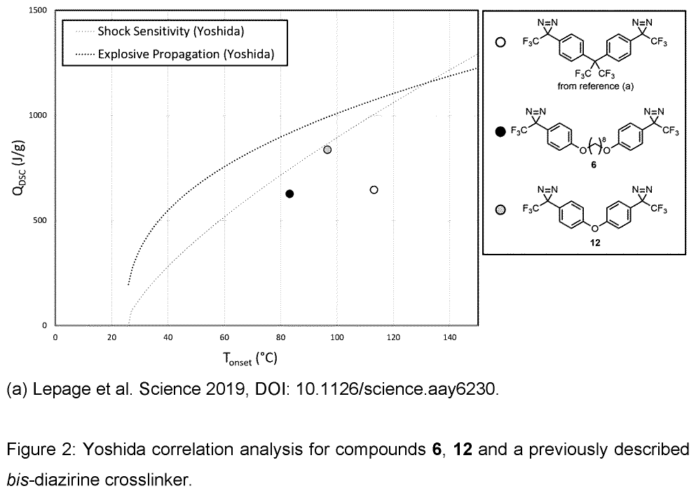

It is also known in the art that the Yoshida correlations can be represented graphically by plotting lines corresponding to the shock sensitivity and explosive propagation thresholds onto a chart of enthalpy of reaction (Q) vs. Tₒₙₛₑₜ. Any processes that fall below these lines are predicted to be non-hazardous. Carrying out this exercise for a representative series of bis-diazirines reveals, as shown in FIG. 2, that bis-ether 6 is well below the two lines, while diaryl ether 12 falls approximately onto the shock sensitivity curve.

From this analysis, we conclude that compound 6 can be viewed as a safe reagent to handle, while compound 12 should be handled with greater caution.

### Example 12: Loading of Crosslinker into UHMWPE Fabric

To further build upon the cyclohexane crosslinking experiment described above, in which bis-ether 6 was shown to have superior efficacy to previously studied molecular crosslinkers, the irreversible crosslinking of ultra-high molecular weight polyethylene (UHMWPE) was studied. For this experiment, compound 6 was directly compared to the first-generation molecular crosslinker described in Lepage et al. Science 2019, DOI: 10.1126/science.aay6230.

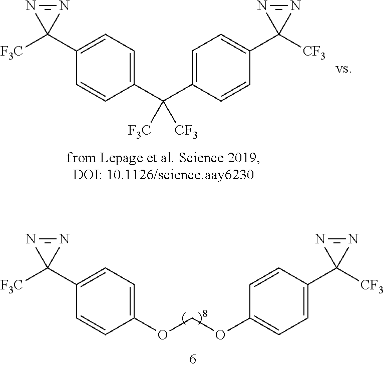

Commercial 75 g/m² UHMWPE fabric was impregnated with either test compound, by placing 1″×1″ pieces of fabric into close-fitting aluminum pans filled with solutions of the desired bis-diazirine in pentane. The concentration of the solution was calculated to correspond to 1.25 wt %, 6.25 wt %, or 12.5 wt % of crosslinker, relative to the mass of fabric being employed in the experiment. The bath was covered with aluminum foil and incubated at room temperature for 30 min. The cover was then removed to allow the pentane to evaporate in a fumehood for 20 min. After pentane evaporation, the impregnated sheets were wrapped in aluminum foil and placed into an oven at 110° C. for 4 h.

Control samples were prepared following an identical procedure, but without adding crosslinker to the pentane bath.

Following thermal curing, the samples were weighed to determine the total mass of reacted crosslinker that was associated with each square of fabric. Each sample was then extracted for 5 min at room temperature using 20 mL of methanol, to remove any reaction products that were not irreversibly attached to the fabric. After drying the treated fabrics in an oven (5 min at 100° C.), each sample was weighed again to determine the mass of reaction products that were lost to the methanol extraction.

| | | | | wt. of | | | | | |
|---|---|---|---|---|---|---|---|---|---|
| | | | | of | | | wt. | | |
| | | wt. | | fabric | wt. | | after | wt. | |
| | nominal | of | wt. | after | gain/ | wt % | extraction | gain/ | wt % |
| | loading | fabric | crosslinker | curing | loss | gain/ | with | loss | gain/ |
| sample | (wt %) | (mg) | (mg) | (mg) | (mg) | loss | MeOH | (mg) | loss |
| control | — | 98.0 | — | 97.7 | −0.3 | −0.31% | 97.2 | −0.5 | −0.51% |
| | | 86.8 | — | 86.5 | −0.3 | −0.35% | 86.1 | −0.4 | −0.46% |
| | | 81.0 | — | 80.8 | −0.2 | −0.25% | 80.3 | −0.5 | −0.62% |
| | | 108.3 | — | 108.1 | −0.2 | −0.18% | 107.8 | −0.3 | −0.28% |
| | | 82.6 | — | 82.3 | −0.3 | −0.36% | 81.9 | −0.4 | −0.49% |
| | | 96.0 | — | 95.8 | −0.2 | −0.16% | 95.5 | −0.3 | −0.31% |
| 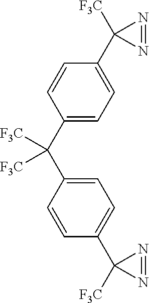 [SMILES: FC(F)(F)C1(c2ccc(C(c3ccc(C4(C(F)(F)F)N=N4)cc3)(C(F)(F)F)C(F)(F)F)cc2)N=N1] | 12.5 6.25 1.25 | 96.0 101.1 103.3 114.2 100.8 99.1 102.1 106.8 103.9 | 12.0 12.6 12.9 7.1 6.3 6.2 1.3 1.3 1.3 | 104.0 110.1 112.6 119.1 104.9 103.2 103.0 107.8 104.9 | 8.0 9.0 9.3 4.9 4.1 4.1 0.9 1.0 1.0 | 8.33% 8.90% 9.00% 4.29% 4.07% 4.14% 0.88% 0.94% 0.96% | 96.0 102.0 102.8 114.8 102.2 100.0 102.0 107.0 104.2 | −8.0 −8.1 −9.8 −4.3 −2.7 −3.2 −1.0 −0.8 −0.7 | −7.69% −7.36% −8.70% −3.61% −2.57% −3.10% −0.97% −0.74% −0.67% |
| 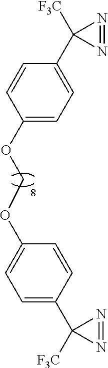 | 12.5 6.25 1.25 | 110.6 96.7 114.1 103.7 101.3 113.5 106.6 94.7 113.9 | 13.8 12.1 14.3 108.0 105.6 118.8 107.7 95.6 114.8 | 120.3 103.6 123.6 108 105.6 118.8 107.7 95.6 114.8 | 9.7 6.9 9.5 4.3 4.3 5.3 1.1 0.9 0.9 | 8.77% 7.14% 8.33% 4.15% 4.24% 4.67% 1.03% 0.95% 0.79% | 120.3 103.4 123.2 107.3 105.1 118.2 107.5 95.5 114.3 | 0.0 −0.2 −0.4 −0.7 −0.5 −0.6 −0.2 −0.1 −0.5 | 0.00% −0.19% −0.32% −0.65% −0.47% −0.51% −0.19% −0.10% −0.44% |

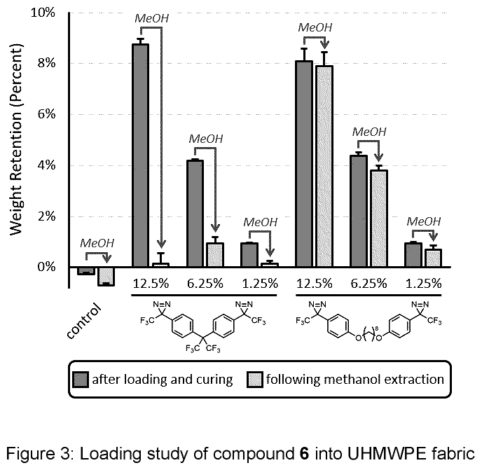

Surprisingly, while both types of bis-diazirines added similar amounts of mass to the fabric upon initial curing, a substantial amount of the reaction products from the first-generation crosslinker were lost following methanol extraction. Correcting for the ‘background’ loss of material at each stage by extraction of the fabric with pentane and methanol, we found that circa 30% of added first-generation crosslinker products were lost at low loadings, circa 60% of first-generation crosslinker was lost at medium loadings, and circa 90% of first-generation crosslinker was lost at high loadings. By contrast, no significant loss of reaction products (relative to control samples) from compound 6 was observed, as shown in FIG. 3.

These data indicate that bis-diazirines based upon aryl ether scaffolds may be superior crosslinking reagents for low-functionality polymers.

What is claimed is:

1. A compound of Formula I: 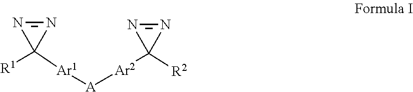
wherein:
A is —X-L-Y—;
R¹ and R² are independently selected from the group consisting of alkyl and cycloalkyl;
Ar¹ and Ar² are independently selected from the group consisting of ortho-, meta- and para-phenylene;
X and Y are independently selected from the group consisting of O and S; and
L is a linear or branched divalent linker selected from the group consisting of saturated aliphatic chains and saturated ethers having from 2 to 20 carbon atoms, and which may optionally include chemically or enzymatically cleavable motifs within the chain, such as esters, silyl ethers, peptides, and the like.

2. A compound of claim 1 wherein Ar¹ and Ar² are each para-phenylene.

3. A compound of claim 2 wherein X and Y are each O.

4. A compound of claim 3 wherein R¹ and R² are each trifluoromethyl.

5. The compound of claim 4 wherein L is (CH₂)₈.

6. The compound of claim 4 wherein L is C₂H₄OSi(iPr)₂OC₂H₄.

7. The compound of claim 4 wherein L is C₂H₄OC(O)OC₂H₄.

8. The compound of claim 4 wherein L is C₂H₄O(CO)₂OC₂H₄.

9. A method of crosslinking a polymer using a compound of Formula I: 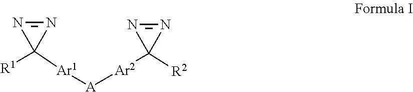
wherein:
A is selected from the group consisting of O, S and —X-L-Y—;
R¹ and R² are independently selected from the group consisting of alkyl and cycloalkyl;
Ar¹ and Ar² are independently selected from the group consisting of ortho-, meta- and para-phenylene;
X and Y are independently selected from the group consisting of O and S; and
L is a linear or branched divalent linker selected from the group consisting of saturated aliphatic chains and saturated ethers having from 2 to 20 carbon atoms. and which may optionally include chemically or enzymatically cleavable motifs within the chain, such as esters, silyl ethers, peptides, and the like.

10. The method of claim 9, wherein A is —X-L-Y—.

11. The method of claim 10, wherein the compound of Formula I is one in which R¹ and R² are CF₃, Ar¹ and Ar² are para-phenylene, X and Y are O, and is L is (CH₂)₈.

12. The method of claim 10, wherein the compound of Formula I is one in which R¹ and R² are CF₃, Ar¹ and Ar² are para-phenylene, X and Y are O, and is L is C₂H₄OSi(iPr)₂OC₂H₄.

13. The method of claim 10, wherein the compound of Formula I is one in which R¹ and R² are CF₃, Ar¹ and Ar² are para-phenylene, X and Y are O, and is L is C₂H₄OC(O)OC₂H₄.

14. The method of claim 10, wherein the compound of Formula I is one in which R¹ and R² are CF₃, Ar¹ and Ar² are para-phenylene, X and Y are O, and is L is C₂H₄O(CO)₂OC₂H₄.

15. The method of claim 9, wherein the compound of Formula I is one in which R¹ and R² are CF₃, Ar¹ and Ar² are para-phenylene, and A is O.

16. The method of claim 9, wherein the crosslinking step is accomplished thermally, photochemically or through the application of an electric field.

17. The method of claim 16 wherein the polymer is a non-functionalized polymer.

18. The method of claim 17 wherein the non-functionalized polymer is selected from the group consisting of polyethylene and polypropylene.

19. The method of claim 18 wherein the non-functionalized polymer is polyethylene.
